(19) United States
(12) Patent Application Publication
(10) Pub. No.: US 2006/0122611 A1
(43) Pub. Date: Jun. 8, 2006

(54) Sternum closure device

(75) Inventors: Pedro Morales, Tuttlingen (DE); Manfred Dworschak, Duerbheim (DE); Leon Eijsman, Amsterdam (NL)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/274,921

(22) Filed: Nov. 14, 2005

Related U.S. Application Data
(63) Continuation of application No. PCT/EP04/05918, filed on Jun. 2, 2004.

(30) Foreign Application Priority Data
Jun. 4, 2003 (DE) 103 26 690.9

(51) Int. Cl. A61B 17/58

(52) U.S. Cl. 606/72

(57) ABSTRACT

In a sternum closure device for fixing two sternum portions to be joined to each other, comprising a first plate-shaped contact element for placement against the inner surface of the sternum, a clamping element fixed on and protruding transversely from the first contact element, a second plate-shaped contact element for placement against the outer side of the sternum, the second contact element being clampable against the first contact element by means of the clamping element passing through the space between the sternum portions, and fixing projections on the first contact element pointing in the direction towards the second contact element, in order to facilitate placement of the sternum closure device, it is proposed that at least one fixing projection on the first contact element be provided with barb-shaped recesses.

This application is a continuation of international application number PCT/EP2004/005918 of Jun. 2, 2004.

The present disclosure relates to the subject matter disclosed in international application number PCT/EP2004/005918 of Jun. 2, 2004 and German application number 103 26 690.9 of Jun. 4, 2003, which are incorporated herein by reference in their entirety and for all purposes.

### BACKGROUND OF THE INVENTION

The invention relates to a sternum closure device for fixing two sternum portions to be joined to each other, comprising a first plate-shaped contact element for placement against the inner surface of the sternum, at least one clamping element fixed on and protruding transversely from the first contact element, a second plate-shaped contact element for placement against the outer side of the sternum, the second contact element being clampable against the first contact element by means of the clamping element passing through the space between the sternum portions, and fixing projections on the first contact element pointing in the direction towards the second contact element.

Such a sternum closure device is described, for example, in U.S. Pat. No. 4,279,248. It serves to fix the two halves of the sternum, which have been severed during an operation, in the pulled-together position relative to each other, so that the two halves can grow together in the desired manner. In the known sternum closure device, the plate-shaped contact elements are provided with tooth-shaped projections with pointed sides, which bite into the sternum and thereby fix the sternum portions securely relative to each other when the two contact elements are clamped against each other. However, these projections only bite into the sternum when the contact elements are clamped against each other. The placement of the contact elements as such is quite complicated, as it must be ensured that the first contact element, which is to be placed against the inner side of the sternum, remains in its contact position. This makes it necessary, in the known device, to fix the clamping elements in the form of threaded rods on a special supporting element.

The object of the invention is to so design a generic sternum closure device that placement of the sternum closure device is facilitated.

### SUMMARY OF THE INVENTION

This object is accomplished in a sternum closure device of the kind described at the outset, in accordance with the invention, in that at least one fixing projection on the first contact element is provided with barb-shaped recesses. In this way, it is possible, prior to clamping together the sternum portions, to securely fix the first contact element on the inner side of one of the two sternum portions, so that the contact element is securely held on this sternum portion until the clamping. The holding is effected by a projection with barbs biting into the sternum portion, when the first contact element is pressed against the inner side of the sternum portion, and fixing the contact element on the inner side of the sternum portion so securely by means of the barbs that the operating surgeon requires no further aids to keep the contact element in this position until the sternum portions are pulled together and the contact elements are subsequently clamped together.

It is advantageous for at least one fixing projection provided with recesses to protrude further in the direction towards the second contact element than other fixing projections. In other words, the fixing projections with barb-shaped recesses are preferably of longer configuration than other fixing projections, so that these penetrate deeper into the sternum tissue when the first contact element is pressed against the sternum.

The barb-shaped recesses may be simple, saw-toothed recesses. They can be microhooks, i.e., the surface or the side edges of the projections are provided with small steps. However, they may also be relatively large recesses, it only being essential that the fixing projections provided with barb-shaped recesses be easy to press into the tissue of the sternum but very difficult to pull out again.

In a first preferred embodiment, provision is made for the first contact element to have only one fixing projection with recesses and a plurality of fixing projections without recesses.

It is advantageous for the fixing projections at ends extending substantially parallel to the space between the sternum portions to be slightly inclined relative to a plane lying perpendicular to the contact surface of the contact element. Owing to this inclination, the fixing projections can clamp the sternum portions against each other when the contact elements are clamped against each other.

To this end, it is, for example, advantageous for the inclined fixing projections to be inclined towards one another on opposite sides.

In principle, the contact elements can be clamped against each other by means of a single clamping element, but it is advantageous for two clamping elements to be secured to the first contact element so as to extend in spaced parallel relation to each other.

The clamping element may be pin-shaped. However, band-shaped or bar-shaped designs are also possible.

It is advantageous for the second contact element to carry at least one elastic tongue which rests against the clamping element and enables displacement of the contact element relative to the clamping element in one direction only. The second contact element can thus be readily displaced along the clamping element against the first contact element, but is then prevented by the elastic tongue or the elastic tongues from moving away from the first contact element again. These tongues may, for example, be formed by radial slots, which emanate from an opening in the second contact element, through which the clamping element passes.

In a modified embodiment, provision is made for a holding member displaceable on the clamping element to be arranged on the side of the second contact element that faces away from the first contact element. The holding member carries at least one elastic tongue which rests against the clamping element and enables displacement of the holding member relative to the clamping element in one direction only. In this case, the elastic tongue is therefore not arranged in the contact element itself, but in a separate holding member, which is displaceable on the clamping element and thereby clamps the second contact element against the first contact element.

In accordance with a preferred embodiment, provision is made for the contact elements to be rectangular.

It is preferable for a fixing projection with barb-shaped recesses to be arranged at the center of the longer side edge of the first contact element.

In a preferred embodiment, the fixing projections on the second contact element are of the same design as the fixing projections on the first contact element. In this case, the second contact element therefore also carries at least one fixing projection with barb-shaped recesses, preferably one fixing projection which is longer than fixing projections without any such recesses.

The following description of preferred embodiments of the invention serves in conjunction with the drawings to explain the invention in greater detail.

### BRIEF DESCRIPTION OF THE DRAWINGS

FIG. 1 shows a schematic front view of two sternum halves with three sternum closure devices arranged along the separating line between the two sternum halves;

FIG. 2 shows a perspective view of a sternum closure device before the contact elements are clamped against each other;

FIG. 3 shows a longitudinal sectional view through a sternum closure device after placement on the two sternum halves and in the clamped state; and

FIG. 4 shows a view of the sternum closure device of FIG. 3 in the direction of arrow A in FIG. 3.

### DETAILED DESCRIPTION OF THE INVENTION

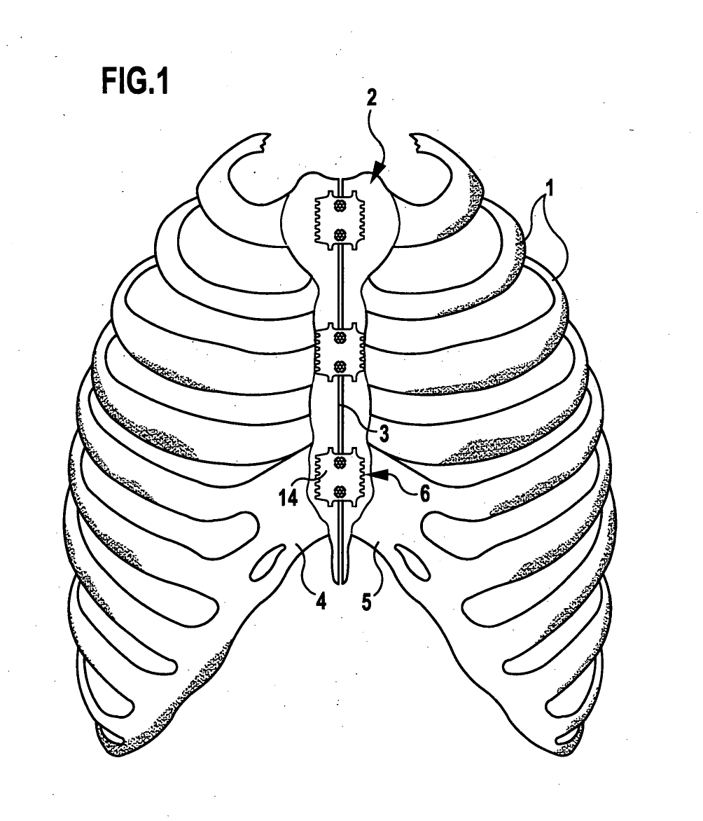

FIG. 1 shows the rib arches 1 of a patient. These are divided along the sternum 2 by a separating cut 3 in order to thereby gain access to the chest cavity. When the operation has been carried out, the two sternum portions 4, must be joined along the separating cut 3. To this end, the two sternum portions 4, 5 are pressed against each other by a tool, not shown in the drawings, for example, by means of fixation forceps, and must be fixed relative to each other in this position.

This fixing is carried out by means of sternum closure devices 6 arranged in spaced relation to one another. Three such sternum closure devices are used in the illustrated embodiment.

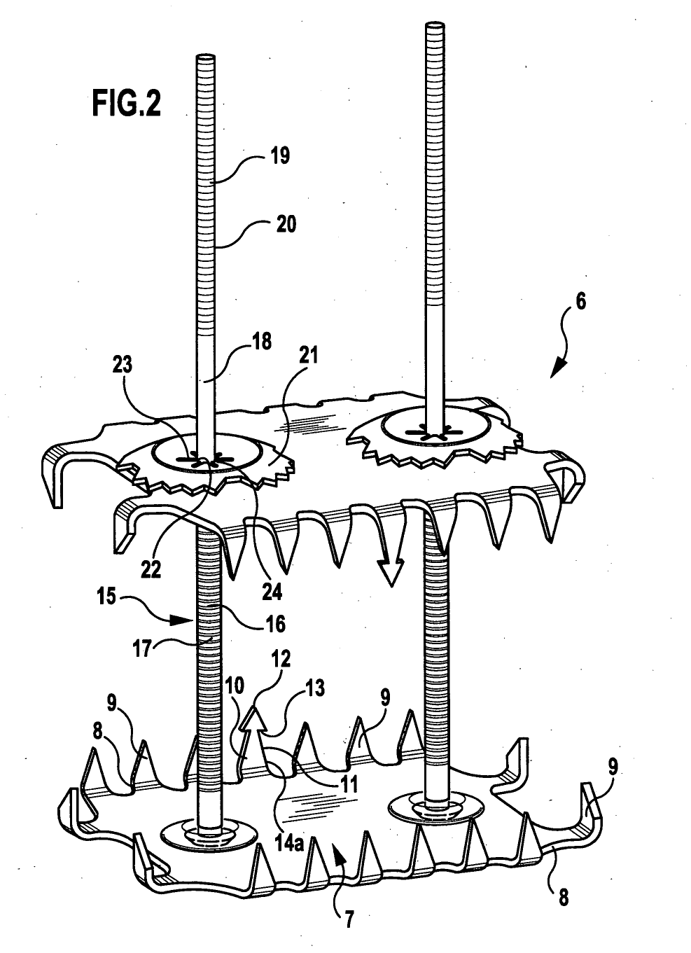

Each sternum closure device 6 is of identical design, which will be explained hereinbelow with reference to FIGS. 2 to 4.

The sternum closure device 6 comprises a rectangular, plate-shaped first contact element 7 with arms 8 protruding from it in all directions. At their free ends, the arms 8 are bent substantially vertically and form laterally bevelled tooth-shaped fixing projections 9 there. Most of the fixing projections narrow continuously towards the tip. Only a single fixing projection 10, which is arranged at the center of the longitudinal side of the contact element 7 and is of longer configuration than the remaining fixing projections 9, has a cross section of approximately arrow shape, so that two recesses 11 are thereby formed on opposite sides. These recesses are delimited at their end facing the tip 12 by an edge 13 extending transversely to the longitudinal direction of the fixing projection 10, and otherwise by an edge 14a which extends at an incline relative to the longitudinal direction of the fixing projection 10, so that the width of the fixing projection 10 decreases towards the tip 12. The recess 11 thus forms a barb. Upon placing the contact element 7 against the inner side of a sternum portion 4, all of the fixing projections 9 and 10 press into the bone and cartilage tissue, and the barb-shaped recess 11 prevents pulling of the fixing projection 10 out of this tissue and thus holds the contact element 7 securely against the inner side of the corresponding sternum portion.

As regards the features described hereinabove, a second contact element is of identical design to the first contact element 7. In the sternum closure 6, it is arranged opposite to the first contact element 7 so that the fixing projections 9 and 10 of the first contact element 7 and of the second contact element 14 face towards one another with their tips.

The first contact element 7 carries two pins 15 extending in parallel spaced relation to each other. These are permanently connected to the first contact element 7, for example, by screw or rivet fastening or by welding. In the section 16 adjacent to the first contact element 7, the pin 15 is provided with circumferential grooves 17 of saw-toothed cross section. This is followed by a smooth section 18 and finally by a further grip section 20 provided with a profile 19.

The two pins 15 pass through openings in the second contact element 14 and carry on the outer side of the second contact element 14 two disc-shaped holding members 21, which also have a central opening 22 for passage of the pin 15 therethrough. Radial cuts 23 emanate from this central opening 22, so that triangular tongues 24 are cut out of the material of the holding member 21. By virtue of appropriate choice of the material of the holding members 21, these tongues are elastically bendable out of their plane and rest with their tips against the circumference of the pin 15. When the holding member 21 is pushed on the pin 15 in the direction towards the first contact element 7, the tongues 24 bend elastically upwards and enable this displacement, but a displacement in the reverse direction is prevented by the upwardly bent tongues 24 becoming wedged relative to the circumference of the pin 15 and biting into it and engaging the circumferential grooves 17, so that, in this way, displacement of the holding member 21 can only take place in one direction, which will be referred to hereinbelow as the clamping direction. Upon displacing the holding members 21 on the corresponding pins 15, these place themselves against the outer side of the second contact element 14 and displace it in the direction towards the first contact element 7, whereby the two contact elements 7 and 14 are clamped against each other and fixed in this position by the holding members 21. The fixing projections 9 and 10 thereupon project into the tissue of the sternum 2 and fix the sternum portions 4 and 5 securely relative to each other.

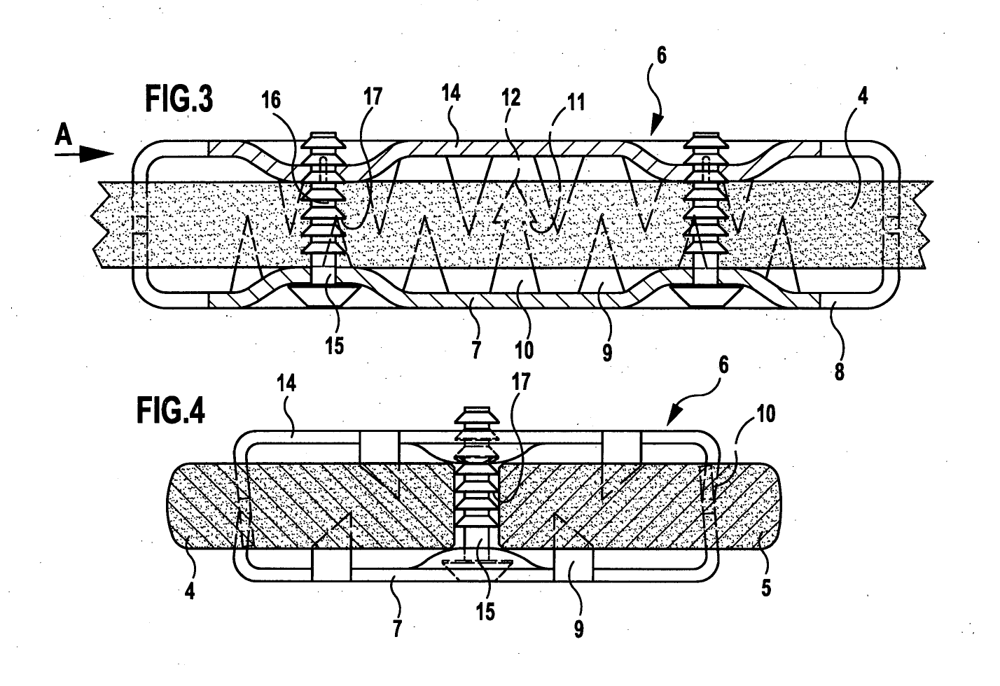

In the embodiment of FIGS. 3 and 4, no holding members 21 are used, but the openings in the second contact element 14, through which the pins 15 pass, are designed like the central opening 22 in the holding member 21, i.e., cuts are provided, which detach elastic tongues from the material of the second contact element 14. In this way, the second contact element 14 can be displaced along the pins 15 in the clamping direction, but not in the opposite direction.

As will be apparent from FIG. 4, some fixing projections 9 are at a slight incline to the clamping direction, so that upon clamping the contact elements 7 and 14 together, a transverse component is exerted on the sternum portions 4, 5 and additionally clamps these to one another.

When putting the sternum closure devices in place, the operating surgeon can pass the first contact elements 7 through into the body cavity between the sternum portions 4, 5, which still maintain a certain distance from each other, and then fix these enduringly on the inner side of a sternum portion by the fixing projection 10 being pressed with the barb-shaped recess 11 into the tissue of the corresponding sternum portion. As this is only necessary on one sternum portion, a corresponding fixing projection 10 with barb-shaped recesses is only arranged on one side of the contact element 7 in the preferred embodiment.

Not only is the first contact element 7 thereby fixed on the sternum portion, but also the pins 15 fixedly connected to it, which are positioned so as to be arranged immediately adjacent to the separating cut 3. The sternum portions are now pressed against each other using a suitable tool, and the second contact element 14 is clamped against the first contact element 7 by a suitable tool. For this purpose, a forceps-type instrument may, for example, be used, which grips the profiled grip section 20 and with a second part simultaneously displaces the holding members 21 or the second contact element 14 in the direction towards the first contact element 7.

After completion of the clamping procedure, the fixing projections 9, 10 of the second contact element 14 also engage the tissue of the sternum 2, and the two sternum portions 4, 5 are enduringly and securely fixed relative to each other. The projecting portions of the pins 15 are removed, and the sternum closure devices remain as implant in the body.

For this reason, it is also advantageous for the sternum closure devices to be made from an absorbable material, so that they can be gradually decomposed after healing. In other cases, however, standard metallic materials which are compatible with the body are used, for example, titanium or titanium alloys.

1. Sternum closure device for fixing two sternum portions to be joined to each other, comprising a first plate-shaped contact element for placement against the inner surface of the sternum, a clamping element fixed on and protruding transversely from the first contact element, a second plate-shaped contact element for placement against the outer side of the sternum, the second contact element being clampable against the first contact element by means of the clamping element passing through the space between the sternum portions, and fixing projections on the first contact element pointing in the direction towards the second contact element, wherein at least one fixing projection on the first contact element is provided with barb-shaped recesses.

2. Sternum closure device in accordance with claim 1, wherein at least one fixing projection provided with recesses protrudes further in the direction towards the second contact element than other fixing projections.

3. Sternum closure device in accordance with claim 1, wherein the first contact element comprises only one fixing projection with recesses and a plurality of fixing projections without recesses.

4. Sternum closure device in accordance with claim 1, wherein at least one fixing projection at ends extending substantially parallel to the space between the sternum portions is slightly inclined relative to a plane lying perpendicular to the contact surface of the contact element.

5. Sternum closure device in accordance with claim 2, wherein at least one fixing projection at ends extending substantially parallel to the space between the sternum portions is slightly inclined relative to a plane lying perpendicular to the contact surface of the contact element.

6. Sternum closure device in accordance with claim 4, wherein the inclined fixing projections are inclined towards one another on opposite sides.

7. Sternum closure device in accordance with claim 5, wherein the inclined fixing projections are inclined towards one another on opposite sides.

8. Sternum closure device in accordance with claim 1, wherein two clamping elements are fixed on the first contact element so as to extend in parallel spaced relation to each other.

9. Sternum closure device in accordance with claim 1, wherein the clamping element is of pin-shaped design.

10. Sternum closure device in accordance with claim 6, wherein the clamping element is of pin-shaped design.

11. Sternum closure device in accordance with claim 1, wherein the second contact element carries elastic tongues which rest against the clamping element and enable displacement of the contact element relative to the clamping element in one direction only.

12. Sternum closure device in accordance with claim 1, wherein a holding member displaceable on the clamping element is arranged on the side of the second contact element facing away from the first contact element and carries elastic tongues which rest against the clamping element and enable displacement of the holding member relative to the clamping element in one direction only.

13. Sternum closure device in accordance with claim 1, wherein the contact elements are of rectangular design.

14. Sternum closure device in accordance with claim 13, wherein a fixing projection with barb-shaped recesses is arranged at the center of the longer side edge of the first contact element.

15. Sternum closure device in accordance with claim 1, wherein the fixing projections on the second contact element are of the same design as on the first contact element.

16. Sternum closure device in accordance with claim 2, wherein the fixing projections on the second contact element are of the same design as on the first contact element.

17. Sternum closure device in accordance with claim 4, wherein the fixing projections on the second contact element are of the same design as on the first contact element.
